(19) United States
(12) Patent Application Publication
(10) Pub. No.: US 2024/0257001 A1
(43) Pub. Date: Aug. 1, 2024

(54) Scheduling Apparatus And Scheduling Method

(71) Applicant: Terumo Kabushiki Kaisha, tokyo (JP)

(72) Inventor: Masatsugu IGARASHI, Kanagawa (JP)

(21) Appl. No.: 18/634,086

(22) Filed: Apr. 12, 2024

Related U.S. Application Data
(63) Continuation of application No. PCT/JP22/45322, filed on Dec. 8, 2022.

(30) Foreign Application Priority Data
Dec. 27, 2021 (JP) 2021-211851

(51) Int. Cl. G06Q 10/0631

(52) U.S. Cl. CPC G06Q 10/063116; G06Q 10/063114

(57) ABSTRACT

A scheduling apparatus includes a process acquisition unit for acquiring an operation process of cell culturing, a condition acquisition unit for acquiring operational conditions including at least information on a working date and time of a user, a process determination unit for determining a timing at which an actual work requiring human labor occurs on the basis of the operation process acquired by the process acquisition unit, and a schedule fixing unit for adjusting a schedule of the operation process in a manner that the operational conditions acquired by the condition acquisition unit harmonize with a determination result of the process determination unit.

### CROSS-REFERENCE TO RELATED APPLICATIONS

The present application is a continuation application of the International Patent Application No. PCT/JP2022/045322 filed Dec. 8, 2022, which designated the U.S. and claims the benefit of priority from Japanese Patent Application No. JP2021-211851 filed Dec. 27, 2021. The entire disclosures of the above-identified applications are incorporated herein by reference.

### FIELD

The present disclosure relates to a scheduling apparatus and a scheduling method for scheduling cell culturing.

### BACKGROUND

A cell culturing device is disclosed in JP 2020-171241A. The cell culturing device is equipped with a bioreactor, a supply unit, a collection unit, and a plurality of flow paths. One portion of the flow paths forms a circulation path together with the bioreactor. The supply unit supplies a cell-containing solution and a culture medium (culturing solution) to the bioreactor. The bioreactor carries out culturing of the cells. During culturing of the cells, one portion of the medium (a first medium) circulates in the circulation path. During culturing of the cells, another portion of the medium (a second medium) is discharged as a waste liquid. The cells that have been cultured are collected by the collection unit.

The process of culturing cells can take several days from start to finish. The operation process often includes actual work that requires human labor. For example, a user may need to attach to a cell culturing device a medical bag containing a medium. In addition, when the medium in the bag runs out during the cell culturing, the user may need to detach the empty bag from the cell culturing device and attach a new bag to the cell culturing device. Further, the quality of the medium may deteriorate if the medical bag is left in a room temperature environment for a long period of time. Therefore, it is preferable to replace the attached bag with a new bag every several days. In addition, when the cell culturing device does not have an automatic control function and the operation of the cell culturing device is to be changed in the middle of the cell culturing, the user needs to control the cell culturing device.

The timing of these different events varies depending on the state of cells, culturing conditions, and the like. For this reason, the actual work cannot be foreseen and makes planning difficult. In the conventional cell culturing operation, the user therefore needs to constantly check the culturing device. As a result, the working time of the user increases and also the burden on the user.

### SUMMARY

The present disclosure provides a scheduling apparatus configured to schedule cell culturing. The scheduling apparatus may include a process acquisition unit configured to acquire an operation process of the cell culturing, a condition acquisition unit configured to acquire operational conditions including at least information on a user working date and time, a process determination unit configured to determine a timing at which an actual work requiring human labor occurs based on the operation process acquired by the process acquisition unit, and a schedule fixing unit configured to fix a schedule of the operation process in a manner that the operational conditions acquired by the condition acquisition unit harmonize with a determination result of the process determination unit.

The operational conditions of the scheduling apparatus may include one or more dates and times at which the user does not work or one or more dates and times at which the user does work, as the information on the user working date and time.

The schedule fixing unit may find out the one or more dates and times at which the user does not work based on the operational conditions and may fix the schedule of the operation process to eliminate overlap between the one or more dates and times at which the user does not work and the timing at which the actual work occurs.

The scheduling apparatus may further include a display unit that may be configured to display the schedule fixed by the schedule fixing unit.

The scheduling apparatus may further include a simulation execution unit configured to simulate the cell culturing and a process creation unit configured to create the operation process based on a result of the simulation. The process acquisition unit may acquire the operation process from the process creation unit.

The scheduling apparatus may further include an input unit configured to input the operational conditions by the user operating the input unit. The condition acquisition unit may acquire the operational conditions input by the input unit.

The actual work may be an attachment operation of attaching a bag or a container containing a liquid for the cell culturing to a cell culturing device that performs the cell culturing.

The input unit may input a capacity of the bag or a capacity of the container, the condition acquisition unit may acquire the capacity of the bag or the capacity of the container input by the input unit, and the process determination unit may determine the timing at which the attachment operation of attaching the bag or the container occurs based on the capacity of the bag or the capacity of the container acquired by the condition acquisition unit.

The scheduling apparatus may further include a medium amount calculation unit. The medium amount calculation unit may be configured to calculate an amount of the medium to be prepared to fill the bag or the container at least on the basis of the operational conditions acquired by the condition acquisition unit and the operation process acquired by the process acquisition unit.

the input unit may be capable of inputting a storage period of the medium. The medium amount calculation unit may calculate the amount of the medium at least on the basis of the operational conditions, the operation process, and the storage period.

The present disclosure provides a scheduling method for scheduling cell culturing. The scheduling method may be executed by using a computer. The scheduling method may include a process acquisition step that includes acquiring an operation process of the cell culturing, a condition acquisition step that includes acquiring operational conditions that includes at least information on a user working date and time, a process determination step that includes determining a timing at which an actual work requiring human labor occurs based on the operation process acquired in the process acquisition step, and a schedule fixing step that includes fixing a schedule of the operation process in a manner that the operational conditions acquired in the condition acquisition step harmonize with a determination result of the process determination step.

Using the scheduling apparatus and/or the scheduling method in accordance with the present invention, it is possible to reduce the burden on the user.

### DRAWINGS

FIG. 1 is a schematic illustrating the configuration of an example cell culturing system in accordance with at least one example embodiment;

FIG. 2 is a schematic illustrating the configuration of an example control unit of a cell culturing device, like the cell culturing device illustrated in FIG. 1, in accordance with at least one example embodiment;

FIG. 3 is a schematic illustrating the configuration of an example scheduling apparatus of a cell culturing device, like the cell culturing device illustrated in FIG. 1, in accordance with at least one example embodiment;

FIG. 4 is a flowchart illustrating an example process for fixing a schedule of a cell culturing operation, like the cell culturing device illustrated in FIG. 1, in accordance with at least one example embodiment;

FIG. 5 is a table showing a calendar displayed by an example display unit, like the display unit illustrated at part of the scheduling apparatus illustrated in FIG. 3, in accordance with at least one example embodiment; and

FIG. 6 is a schematic illustrating the configuration of another example scheduling apparatus of a cell culturing device illustrated in FIG. 1, in accordance with at least one example embodiment.

### DETAILED DESCRIPTION

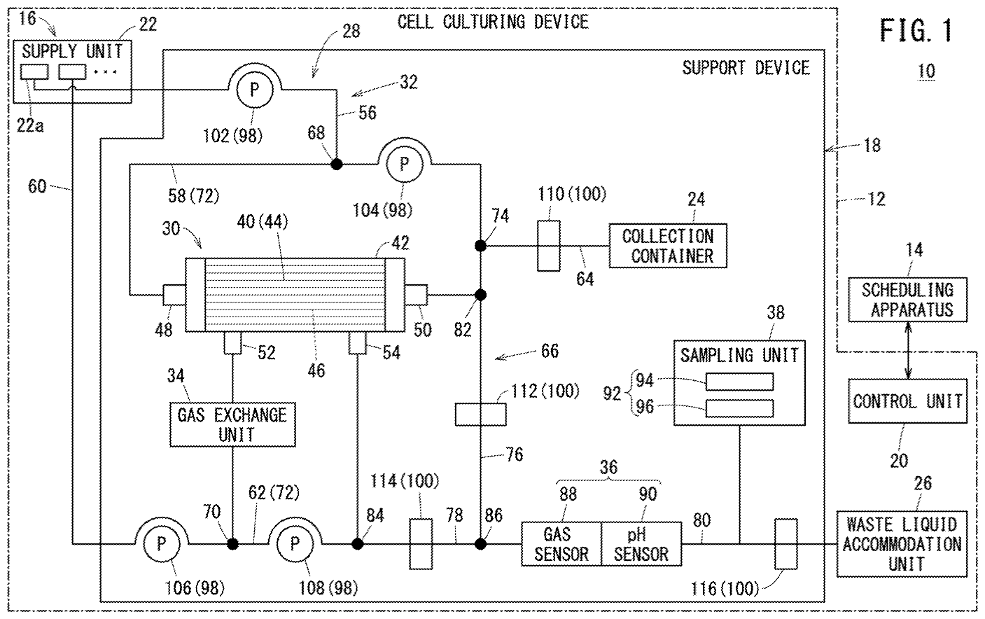

FIG. 1 is a schematic illustrating a configuration of an example cell culturing system 10. The cell culturing system 10 may be configured culture (propagate) within a culture medium cells that have been separated from living tissue. In at least one example embodiment, the cells used in the cell culturing system 10 may include adherent cells. In at least one example embodiment, the cells used in the cell culturing system 10 may be planktonic cells. The cells used in the cell culturing system 10 may include, for example, embryonic stem (ES) cells, induced pluripotent stem (iPS) cells, mesenchymal stem cells, and the like. The cells used in the cell culturing system 10 are not limited to the cell types described above.

The cell culturing system 10 may include a cell culturing device 12 and a scheduling apparatus 14. The cell culturing device 12 may include a cell culturing circuit 16, a support device 18, and a control unit 20. A liquid may flow through the cell culturing circuit 16. The liquid may include, for example, a cell solution, a culture medium, a cleaning solution, a stripping solution, or any combination thereof.

The cell solution may include a solution containing cells. The culture medium may include a culturing solution selected to cause the cells to propagate. The culture medium may be selected depending on the cells to be cultured. In at least one example embodiment, the culture medium may include a minimum essential media (MEM). The cleaning solution may be selected to clean the interior of the cell culturing circuit 16. In at least one example embodiment, the cleaning solution may include, for example, water, a buffer solution, a physiological saline solution or the like, or any combination thereof. The buffer solution may include, for example, phosphate buffered saline (PBS), tris-buffered saline (TBS), and/or the like. The stripping solution may be selected to strip the cells from a later-described bioreactor 30 of the cell culturing circuit 16. The stripping solution may include, for example, trypsin, an ethylenediaminetetraacetic acid (EDTA) solution, or any combination thereof. The culture medium, the cleaning solution, and the stripping solution are not limited to the liquids described above.

The cell culturing circuit 16 may be discarded after a single use thereof. For example, the cell culturing circuit 16 may be discarded each time a predetermined number of cells have been cultured. The cell culturing circuit 16 may be a disposable product. The cell culturing circuit 16 may include a supply unit 22, a collection container 24, a waste liquid accommodation unit 26, and a culturing body 28.

The supply unit 22 may be configured to supply at least one of the cell solution, the culture medium, the cleaning solution, the stripping solution, and the like to the culturing body 28. In at least one example embodiment, the supply unit 22 may include a plurality of bags 22a in which each liquid is separately contained. Each bag 22a may include a medical bag. The medical bag may be formed by molding a soft resin material into a bag shape. In at least one example embodiment, the supply unit 22 may include a plurality of tanks or the like made of a hard material.

The collection container 24 may be configured to collect the cells that are cultured in the culturing body 28. The waste liquid accommodation unit 26 may be configured to accommodate the waste liquid that may be generated in the culturing body 28. In at least one example embodiment, the collection container 24 and the waste liquid accommodation unit 26 may each include a medical bag. The medical bags may be obtained by molding a soft resin material into a bag-like shape. In at least one example embodiment, the collection container 24 and the waste liquid accommodation unit 26 may each include a tank or the like constituted by a hard material.

The culturing body 28 may include a bioreactor 30, flow paths 32, a gas exchange unit 34, a sensor unit 36, and a sampling unit 38.

The bioreactor 30 may include a plurality of hollow fiber membranes 40 and a cylindrical housing 42. The plurality of hollow fiber membranes 40 may be accommodated inside the housing 42. A first end part of the respective hollow fiber membranes 40 may be fixed to a first end part of the housing 42. A second end part of the respective hollow fiber membranes 40 may be fixed to a second end part of the housing 42. In at least one example embodiment, the respective hollow fiber membranes 40 may include a polymer material.

The bioreactor 30 may include a first region 44 and a second region 46. The first region 44 may be defined by inner holes of the plurality of hollow fiber membranes 40. The second region 46 may be defined by a space between an inner peripheral surface of the housing 42 and outer peripheral surfaces of the plurality of hollow fiber membranes 40. The hollow fiber membranes 40 may include a plurality of pores. The first region 44 and the second region 46 may communicate with each other through the plurality of pores. The diameter of the pores may be of a size that allows low molecular-weight compounds (for example, water, ions, oxygen, lactic acid, etc.) to pass therethrough, while preventing the passage of high molecular-weight compounds (for example, cells) therethrough. An average diameter of the respective pores may be greater than or equal to 0.005 micrometers and less than or equal to 10 micrometers.

The housing 42 may include a first inlet port 48, a first outlet port 50, a second inlet port 52, and a second outlet port 54. The first inlet port 48 may be installed at one end of the housing 42. The first inlet port 48 may be in communication with the first region 44 via an inlet positioned at one end of the plurality of hollow fiber membranes 40. The first outlet port 50 may be installed at another end of the housing 42. The first outlet port 50 may be in communication with the first region 44 via an outlet positioned at the other end of the plurality of hollow fiber membranes 40.

The second inlet port 52 and the second outlet port 54 may be installed on an outer peripheral surface of the housing 42. The second inlet port 52 may be positioned between a center of the housing 42 and the first inlet port 48 in the longitudinal direction of the housing 42. The second outlet port 54 may be positioned between the center of the housing 42 and the first outlet port 50 in the longitudinal direction of the housing 42. The second inlet port 52 and the second outlet port 54 may both be in communication with the second region 46.

The flow paths 32 may include a plurality of tubes through which the liquids flow. In at least one example embodiment, the plurality of tubes may include a soft resin material. The flow paths 32 may include a first supply flow path 56, a first circulation flow path 58, a second supply flow path 60, a second circulation flow path 62, a collection flow path 64, and a waste liquid flow path 66. One end of the first supply flow path 56 may be connected to the supply unit 22. The supply unit 22 may supply the cell solution, the culture medium, the cleaning solution, and the stripping solution to the first supply flow path 56. The supply unit 22 may supply the cell solution, culture medium, the cleaning solution, and the stripping solution to the first supply flow path 56 individually at predetermined points in time. Another end of the first supply flow path 56 may be connected to a first merging section 68 within the first circulation flow path 58.

The first merging section 68 may be positioned in an intermediate portion in the direction in which the first circulation flow path 58 extends. One end of the first circulation flow path 58 may be connected to the first inlet port 48. Another end of the first circulation flow path 58 may be connected to the first outlet port 50. The first circulation flow path 58 may be in communication with the inner holes (the first region 44) of the plurality of hollow fiber membranes 40.

One end of the second supply flow path 60 may be connected to the supply unit 22. The supply unit 22 may be configured to supply the culture medium and the cleaning solution individually at predetermined points in time to the second supply flow path 60. Another end of the second supply flow path 60 may be connected to a second merging section 70 within the second circulation flow path 62.

The second merging section 70 may be positioned in an intermediate portion in the direction in which the second circulation flow path 62 extends. One end of the second circulation flow path 62 may be connected to the second inlet port 52. Another end of the second circulation flow path 62 may be connected to the second outlet port 54. The second circulation flow path 62 may be in communication with a space (the second region 46) between the plurality of hollow fiber membranes 40 and the housing 42. The first circulation flow path 58 and the second circulation flow path 62 may be collectively referred to as “circulation flow paths 72”.

The collection flow path 64 may extend from the first circulation flow path 58. One end of the collection flow path 64 may be connected to a collection branching section 74 within the first circulation flow path 58. The collection branching section 74 may be positioned between the first merging section 68 and the first outlet port 50 in the first circulation flow path 58. Another end of the collection flow path 64 may be connected to the collection container 24.

The waste liquid flow path 66 may enable the liquid discarded from the circulation flow paths 72 to flow therethrough. The waste liquid flow path 66 may include a first waste liquid flow path 76, a second waste liquid flow path 78, and a third waste liquid flow path 80. The first waste liquid flow path 76 may extend from the first circulation flow path 58. One end of the first waste liquid flow path 76 may be connected to a first branching section 82 within the first circulation flow path 58.

The first branching section 82 may be positioned between the first outlet port 50 and the collection branching section 74 within the first circulation flow path 58. The second waste liquid flow path 78 may extend from the second circulation flow path 62. One end of the second waste liquid flow path 78 may be connected to a second branching section 84 within the second circulation flow path 62. The second branching section 84 may be positioned between the second merging section 70 and the second outlet port 54 within the second circulation flow path 62. Another end of the first waste liquid flow path 76 and another end of the second waste liquid flow path 78 may be connected together mutually at an intermediate merging section 86. One end of the third waste liquid flow path 80 may be connected at the intermediate merging section 86 to the first waste liquid flow path 76 and the second waste liquid flow path 78. Another end of the third waste liquid flow path 80 may be connected to the waste liquid accommodation unit 26.

The gas exchange unit 34 may be installed within the second circulation flow path 62 between the second merging section 70 and the second inlet port 52. The gas exchange unit 34 may allow a gas having predetermined components to pass through the liquid (the culture medium) that flows through the second circulation flow path 62. The gas used in the gas exchange unit 34 may include, for example, components therein that are similar to those of natural air. For example, the gas used in the gas exchange unit 34 may include nitrogen, oxygen, and carbon dioxide. In at least one example embodiment, the gas may include, for example, 75% nitrogen, 20% oxygen, and 5% carbon dioxide by volume.

The sensor unit 36 may be installed in the third waste liquid flow path 80. The sensor unit 36 may include a gas sensor 88 and a pH sensor 90. The gas sensor 88 may be configured to measure a gas concentration of the liquid that flows through the third waste liquid flow path 80. In at least one example embodiment, the gas sensor 88 may include an oxygen sensor and a carbon dioxide sensor. The oxygen sensor may be configured to measure an oxygen concentration of the liquid that flows through the third waste liquid flow path 80. The carbon dioxide sensor may be configured to measure a carbon dioxide concentration of the liquid that flows through the third waste liquid flow path 80. The pH sensor 90 may be configured to measure a pH (hydrogen ion exponent) of the liquid that flows through the third waste liquid flow path 80. The gas sensor 88 and/or the pH sensor 90 may output measurement results to the control unit 20.

The sampling unit 38 may be connected to a portion within the third waste liquid flow path 80 between the sensor unit 36 and the waste liquid accommodation unit 26. The sampling unit 38 may be configured to extract one portion of the liquid that flows through the third waste liquid flow path 80 and to measure the components contained in the liquid. In at least one example embodiment, the sampling unit 38 may include a biosensor 92, a flow path, and the like.

The biosensor 92 may include, for example, a glucose sensor 94 and a lactic acid sensor 96. The glucose sensor 94 may be configured to measure a glucose concentration of the liquid extracted from the third waste liquid flow path 80. The lactic acid sensor 96 may be configured to measure a lactic acid concentration of the liquid extracted from the third waste liquid flow path 80. Each of the glucose sensor 94 and the lactic acid sensor 96 may output measurement results to the control unit 20.

The cell culturing circuit 16 may be set in the support device 18. The support device 18 may include a cassette that supports the cell culturing circuit 16. The support device 18 may be a reusable product that is capable of being used a plurality of times.

The support device 18 may be equipped with a plurality of pumps 98 and a plurality of clamps 100. Each of the plurality of pumps 98 may impart a flowing force to the liquids inside the flow paths 32 by squeezing the wall parts of the flow paths 32. Each of the plurality of pumps 98 may include a pressing member (not shown). The pressing member may include, for example, a rotating member and a plurality of pressing rollers. The plurality of pressing rollers may be attached to an outer circumferential portion of the rotating member. The plurality of pressing rollers may be arranged at intervals with spaces left therebetween in the circumferential direction of the rotating member. Each of the pressing rollers may rub against the outer surfaces of the wall parts of the flow paths 32.

The plurality of pumps 98 may include a first supply pump 102, a first circulation pump 104, a second supply pump 106, and a second circulation pump 108. Moreover, as illustrated in FIG. 1, a state in which the cell culturing circuit 16 is set in the support device 18 may be simply referred to as a “set state”.

In the set state, a portion of the first supply flow path 56 may be installed on the first supply pump 102. The first supply pump 102 may impart a flowing force to the liquid inside the first supply flow path 56 in a direction from the supply unit 22 toward the first circulation flow path 58.

In the set state, a portion of the first circulation flow path 58 may be installed on the first circulation pump 104. The first circulation pump 104 may impart a flowing force to the liquid inside the first circulation flow path 58 in a direction from the first outlet port 50 toward the first inlet port 48. Moreover, the first circulation pump 104 may impart a flowing force to the liquid inside the first circulation flow path 58 in a direction from the first inlet port 48 toward the first outlet port 50.

In the set state, a portion of the second supply flow path 60 may be installed on the second supply pump 106. The second supply pump 106 may impart a flowing force to the liquid inside the second supply flow path 60 in a direction from the supply unit 22 toward the second circulation flow path 62.

In the set state, a portion of the second circulation flow path 62 may be installed on the second circulation pump 108. The second circulation pump 108 may impart a flowing force to the liquid inside the second circulation flow path 62 in a direction from the second outlet port 54 toward the second inlet port 52. Moreover, the second circulation pump 108 may impart a flowing force to the liquid inside the second circulation flow path 62 in a direction from the second inlet port 52 toward the second outlet port 54.

The plurality of clamps 100 may be configured to close the flow paths 32 by pressing the outer surfaces toward the inner surfaces of the flow paths 32, the plurality of clamps 100 may serve as on/off valves. The plurality of clamps 100 may include a collection clamp 110, a first waste liquid clamp 112, a second waste liquid clamp 114, and a third waste liquid clamp 116.

In the set state, a portion of the collection flow path 64 may be installed in the collection clamp 110. The collection clamp 110 may open and close the collection flow path 64. In the set state, a portion of the first waste liquid flow path 76 may be installed in the first waste liquid clamp 112. The first waste liquid clamp 112 may open and close the first waste liquid flow path 76. In the set state, a portion of the second waste liquid flow path 78 may be installed in the second waste liquid clamp 114. The second waste liquid clamp 114 may open and close the second waste liquid flow path 78. In the set state, a portion of the third waste liquid flow path 80 may be installed in the third waste liquid clamp 116. The third waste liquid clamp 116 may open and close the third waste liquid flow path 80.

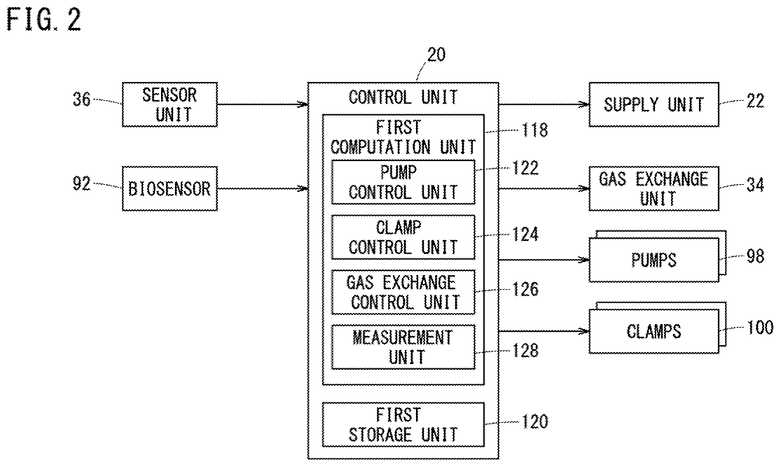

FIG. 2 is a diagram showing the configuration of the control unit 20 of the cell culturing device 12. The control unit 20 may include a first computation unit 118, a first storage unit 120, and various drive circuits (not shown).

The first computation unit 118 may include a processing circuit. The processing circuit may be a processor such as a CPU or the like. The processing circuit may be an integrated circuit such as an ASIC, an FPGA, or the like. The processor may be capable of executing various processes by executing programs stored in the first storage unit 120. The first computation unit 118 may function as a pump control unit 122, a clamp control unit 124, a gas exchange control unit 126, and a measurement unit 128. At least a portion from among the processes may be performed by an electronic circuit including a discrete device.

The pump control unit 122 may control each of the plurality of pumps 98. For example, the pump control unit 122 outputs command signals to a pump drive circuit (not shown). The pump drive circuit may supply power to each of the plurality of pumps 98 in accordance with the command signals from the pump control unit 122. The clamp control unit 124 may control the plurality of clamps 100. For example, the clamp control unit 124 outputs command signals to a clamp drive circuit (not shown). The clamp drive circuit may supply power to each of the plurality of clamps 100 in accordance with the command signals from the clamp control unit 124. The gas exchange control unit 126 may control the gas exchange unit 34. For example, the gas exchange control unit 126 outputs command signals to a gas exchanger drive circuit (not shown). The gas exchanger drive circuit may supply electrical power to the gas exchange unit 34 in accordance with the command signals from the gas exchange control unit 126. The measurement unit 128 may acquire the measurement results from each of the gas sensor 88, the pH sensor 90, the glucose sensor 94, and the lactic acid sensor 96. The measurement unit 128 may cause the first storage unit 120 to store the acquired measurement results.

The first storage unit 120 may include a volatile memory and a non-volatile memory. In at least one example embodiment, the volatile memory may include a RAM or the like. The volatile memory may be used as a working memory of the processor. The volatile memory may temporarily store data and the like required for carrying out processing or computations. In at least one example embodiment, the non-volatile memory may include a ROM, a flash memory, or the like. Such a non-volatile memory may be used as a storage memory. Programs, tables, and maps, etc. may be stored in the non-volatile memory. At least a portion of the first storage unit 120 may be provided in the above-described processor, the integrated circuit, or the like.

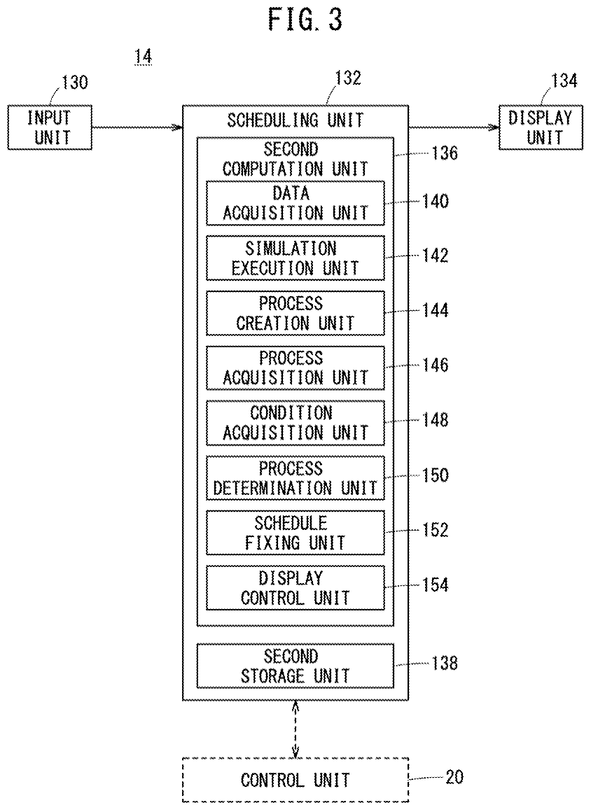

FIG. 3 is a diagram illustrating the configuration of the scheduling apparatus 14. The scheduling apparatus 14 may include an input unit 130, a scheduling unit 132, and a display unit 134. A personal computer, a smart phone, a tablet, or the like may be used as the scheduling apparatus 14.

The input unit 130 may include a human-machine interface such as a keyboard, a mouse, a touch pad, or the like. Further, the input unit 130 may include a human-machine interface that is integrated with the display unit 134, as in the form of a touch panel. The input unit 130 may be capable of inputting data to the scheduling unit 132 corresponding to operations performed by the user.

The scheduling unit 132 may include a second computation unit 136 and a second storage unit 138. The first computation unit 118 and the first storage unit 120 may also be used as the second computation unit 136 and the second storage unit 138. Stated otherwise, the control unit 20 of the cell culturing device 12 may be used as the scheduling unit 132. The second computation unit 136 may include a processing circuit. The processing circuit may be a processor such as a CPU or the like. The processing circuit may be an integrated circuit such as an ASIC, an FPGA, or the like. The processor may be capable of executing various processes by executing programs stored in the second storage unit 138. The second computation unit 136 may function as a data acquisition unit 140, a simulation execution unit 142, a process creation unit 144, a process acquisition unit 146, a condition acquisition unit 148, a process determination unit 150, a schedule fixing unit 152, and a display control unit 154. At least a portion from among the processes may be performed by an electronic circuit including a discrete device.

The data acquisition unit 140 may acquire data from the exterior of the second computation unit 136. The data acquired by the data acquisition unit 140 may be used when a simulation of the cell culturing is executed. For example, the data acquisition unit 140 may be capable of acquiring data from the input unit 130. Further, the data acquisition unit 140 may be capable of acquiring data from the second storage unit 138. Further, the data acquisition unit 140 may be capable of acquiring data from a device (the control unit 20 or the like) specified by the input unit 130.

The simulation execution unit 142 may use the data acquired by the data acquisition unit 140 and thereby simulates the cell culturing by the cell culturing device 12. The simulation execution unit 142 may cause the second storage unit 138 to store the simulation result.

The process creation unit 144 may create an operation process of the cell culturing based on the result of the cell culturing simulation. The operation process may be an operation plan of “when and what to do”. The operation may include actual work which requires the user's manual operation, for example, change of the flow rates of the pumps 98, replacement of the bags 22a, and the like. As the operation process, for example, an execution timing may be set using a start time of the cell culturing as a starting point for each operation to be executed.

The process acquisition unit 146 may acquire the operation process. For example, the process acquisition unit 146 may acquire the operation process from the process creation unit 144 or may acquire a preset operation process from the second storage unit 138. In at least one example embodiment, the second storage unit 138 may store the operation process created by the user operating the input unit 130.

The condition acquisition unit 148 may acquire data indicating the operational conditions from the exterior of the second computation unit 136. The data acquired by the condition acquisition unit 148 may be used when creating the schedule of the operation process. For example, the condition acquisition unit 148 may be capable of acquiring data indicating the operational conditions from the input unit 130. The operational conditions acquired from the input unit 130 may include one or more dates and times at which the user does not work, one or more dates and times at which the user does work, the capacity of the bag 22a, and the like.

The process determination unit 150 may determine a timing at which an actual work requiring human labor occurs based on the operation process acquired by the process acquisition unit 146, the capacity of the bag 22a acquired by the condition acquisition unit 148, and the like. The timing at which the actual work occurs may be indicated by, for example, the execution timing using the start time of the cell culturing as the starting point. The determination criteria of the actual work may be stored in advance in the second storage unit 138.

The schedule fixing unit 152 may be configured to fix the schedule of the operation process so that the operational conditions acquired by the condition acquisition unit 148 and the determination result of the process determination unit 150 do not conflict with each other. For example, when the operational conditions include one or more dates and times at which the user does not work, the schedule fixing unit 152 may fix the schedule of the operation process so that the one or more dates and times at which the user does not work do not coincide with the timing at which the actual work occurs. On the other hand, when the operational conditions include one or more dates and times at which the user works, the schedule fixing unit 152 may determine one or more dates and times at which the user does not work and may fix the schedule of the operation process so that the determined one or more dates and times do not coincide with the timing at which the actual work occurs.

The display control unit 154 may cause the display unit 134 to display various screens. For example, the display control unit 154 may be capable of displaying the data stored in the second storage unit 138 on the display unit 134. The display control unit 154 may be capable of displaying the operation schedule fixed by the schedule fixing unit 152 on the display unit 134. When the schedule is not changeable, the display control unit 154 may display information indicating that the schedule cannot be changed on the display unit 134.

The second storage unit 138 may include a volatile memory and a non-volatile memory. In at least one example embodiment, the volatile memory may include a RAM or the like. The volatile memory may be used as a working memory of the processor. The volatile memory may temporarily store data and the like required for carrying out processing or computations. In at least one example embodiment, the non-volatile memory may include a ROM, a flash memory, or the like. Such a non-volatile memory may be used as a storage memory. Programs, tables, and maps, etc. may be stored in the non-volatile memory. The non-volatile memory may store a simulation program that is executed by the simulation execution unit 142. The non-volatile memory may store a program for fixing the operation schedule. Further, the non-volatile memory may store a calendar. At least a portion of the second storage unit 138 may be provided in the above-described processor, the integrated circuit, or the like.

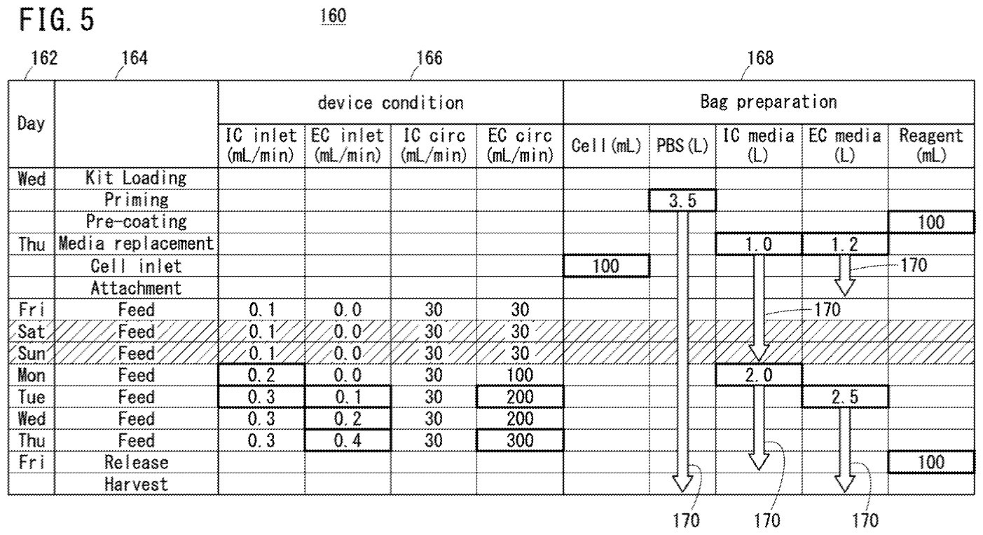

The display unit 134 includes a human-machine interface such as a display or the like. Further, the display unit 134 may include a human-machine interface that is integrated with the input unit 130, for example, in the form of a touch panel. The display unit 134 may be capable of displaying the various screens. For example, as illustrated in FIG. 5, a cell culturing calendar 160 may be displayed.

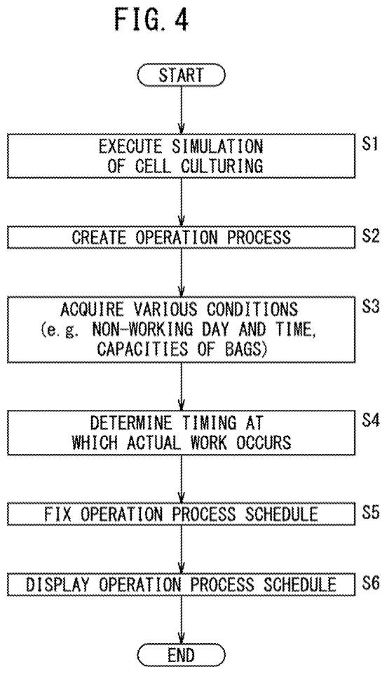

FIG. 4 is a flow chart illustrating a process flow of a series of processes for fixing a schedule of the cell culturing operation. Before starting the series of processes shown in FIG. 4, the user may input each item of data using the input unit 130. The user may input each item of data to be used in the simulation of the cell culturing. The user may input data of various conditions used for creating the operation schedule. For example, the user may input the date and time when actual work is not performed, the capacity of each bag 22a is to be attached to the cell culturing device 12, and the like. The date and time when the user does not perform the actual work may be a date and time when the user cannot perform the actual work, a date and time when the user does not want to perform the actual work, or the like. Each item of data may be stored in the second storage unit 138.

In step S1, the simulation execution unit 142 may execute the simulation of the cell culturing. The data acquisition unit 140 may acquire each item of data from the second storage unit 138. The simulation execution unit 142 may execute the simulation using each item of data acquired by the data acquisition unit 140. The simulation execution unit 142 may cause the second storage unit 138 to store the simulation result. As the simulation result, changes in the operation of each pump 98, changes in the consumption rate of each liquid, and the like may be stored.

In step S2, the process creation unit 144 may create the operation process of the cell culturing based on the simulation result of the cell culturing.

In step S3, the condition acquisition unit 148 may acquire, for example, from the second storage unit 138, data of various conditions (e.g., a date and time when actual work is not performed, a capacity of each bag 22a, and the like) to be used to create an operation schedule.

In step S4, the process determination unit 150 may determine the timing at which the actual work occurs based on the operation process. For example, the operation process may include information indicating a timing at which the flow rate of each medium is changed. When the cell culturing device 12 automatically controls each pump 98, the user may not need to control each pump 98. On the other hand, when the cell culturing device 12 does not automatically control each pump 98, the user may need to control each pump 98 to change the flow rate of the medium. The process determination unit 150 may determine the timing at which the flow rate of each medium is changed as the timing at which the actual work occurs. In addition, the operation process may include information indicating a timing at which each liquid is used and information indicating an amount of each liquid to be consumed. If the amount of liquid to be consumed exceeds the capacity of the bag 22a, replacement (attachment) of the bag 22a may be required. The process determination unit 150 may determine each of the timing at which the liquid is used and the timing at which the bag 22a is replaced, as the timing at which the actual work occurs.

In step S5, the schedule fixing unit 152 may fix the schedule of the operation process. The schedule fixing unit 152 may fix the schedule of the operation process so that the date and time when the user does not work does not coincide with the timing at which the actual work occurs. For example, the schedule fixing unit 152 may select a timing at which no actual work occurs from the operation schedule. The schedule fixing unit 152 may adjust the timing at which the actual work does not occur to the date and time when the user does not work. Furthermore, the schedule fixing unit 152 may fix the schedule of all the operation processes on the basis of the operation performed on the date and time when the user does not work. The same may apply to a case where a plurality of dates and times at which the user does not work are designated.

In step S6, the display control unit 154 may display the operation schedule fixed by the schedule fixing unit 152. The display unit 134 may display the calendar 160 shown in FIG. 5 in response to an instruction from the display control unit 154. The calendar 160 is an example of a visualized operation schedule. The calendar 160 may include a working day column 162, an operation name column 164, a device condition column 166, and a bag preparation column 168.

The scheduled working days may be indicated in the working day column 162. In FIG. 5, days of the week may be indicated as the scheduled working days. In addition, the working day column 162 may be divided not by day but by hour. In the operation name column 164, the names of operations to be performed in the cell culturing device 12 for each day may be indicated.

The flow rate of the medium for each day may be indicated in the device condition column 166. In FIG. 5, the flow rates of the medium in the four flow paths 32 (“IC inlet”, “EC inlet”, “IC circ”, and “EC circ”) may be shown in the device condition column 166. “IC inlet” may be the flow rate of the medium in the first supply flow path 56. The flow rate indicated by “IC inlet” may be controlled by the first supply pump 102. “EC inlet” may be the flow rate of the medium in the second supply flow path 60. The flow rate indicated by “EC inlet” may be controlled by the second supply pump 106. “IC circ” may be the flow rate of the medium in the first circulation flow path 58. The flow rate indicated by “IC circ” may be controlled by the first circulation pump 104. “EC circ” may be the flow rate of the medium in the second circulation flow path 62. The flow rate indicated by “EC circ” may be controlled by the second circulation pump 108.

In the bag preparation column 168, the liquid used in the cell culturing device 12 and the amount of the liquid to be used may be indicated. In FIG. 5, five types of liquids (“Cell”, “PBS”, “IC media”, “EC media”, and “Reagent”) are shown. Each liquid may be contained in a bag 22a. “Cell” may represent the cell solution. According to FIG. 5, the operation of attaching the bag 22a containing 100 mL of the cell solution to the cell culturing device 12 may occur on the first Thursday. “PBS” is a buffer solution as described above. According to FIG. 5, the operation of attaching the bag 22a containing 3.5 L of the buffer solution to the cell culturing device 12 may occur on the first Wednesday. “IC media” may be a medium to be caused to flow through the first supply flow path 56 and the first circulation flow path 58. According to FIG. 5, the operation of attaching a bag 22a containing 1 L of the medium to the cell culturing device 12 may occur on the first Thursday. Further, an operation of attaching a bag 22a containing 2 L of the medium to the cell culturing device 12 may occur on the second Monday. “EC media” may be a medium to be caused to flow through the second supply flow path 60 and the second circulation flow path 62. According to FIG. 5, the operation of attaching a bag 22a containing 1.2 L of the medium to the cell culturing device 12 may occur on the first Thursday. Further, an operation of attaching a bag 22a containing 2.5 L of the medium to the cell culturing device 12 may occur on the second Tuesday. “Reagent” may be another liquid such as the cleaning solution, the stripping solution and the like. According to FIG. 5, an operation of attaching a bag 22a containing 100 mL of the reagent to the cell culturing device 12 may occur on the first Wednesday. In addition, an operation of attaching a bag 22a containing 100 mL of a reagent (stripping agent) to the cell culturing device 12 may occur on the second Friday. Each arrow 170 shown in the bag preparation column 168 may mean that the state in which the bag 22a is attached to the cell culturing device 12 is maintained.

In response to an instruction from the display control unit 154, the display unit 134 may highlight a cell in which an actual work occurs in the device condition column 166 and the bag preparation column 168. In FIG. 5, the cells in which actual works occur may be displayed with bold frames. In addition, in response to an instruction from the display control unit 154, the display unit 134 may highlight a row of the day on which the user does not work in the calendar 160. In FIG. 5, the rows of the days on which the user does not work may be shaded. As shown in FIG. 5, in the calendar 160, the bold framed cells and the shaded rows do not overlap with each other.

In various embodiments, further functions may be added to the scheduling apparatus 14 illustrated in FIG. 3.

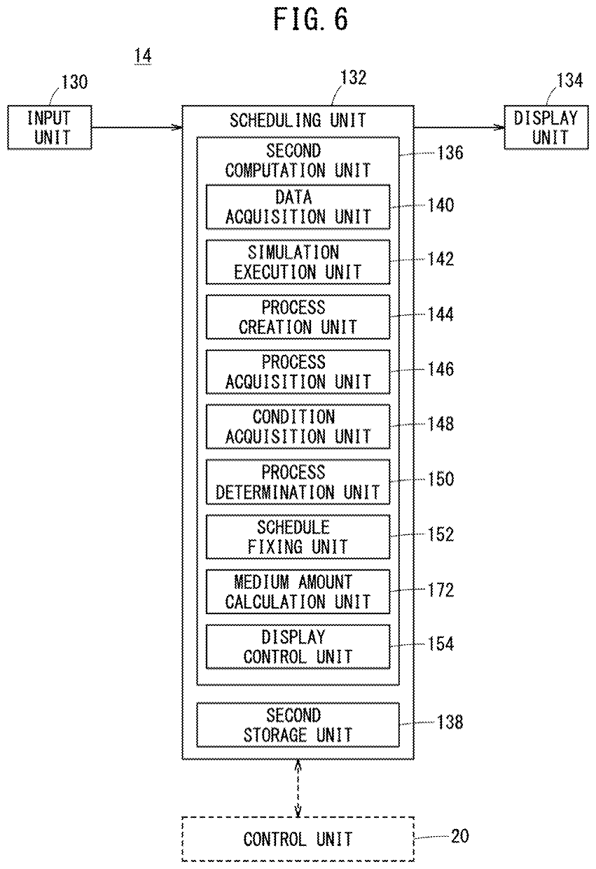

For example, FIG. 6 is a diagram showing the configuration of the scheduling apparatus 14 according to another embodiment. In FIG. 6, the same components as those in FIG. 3 are denoted by the same reference numerals. In the scheduling apparatus 14 shown in FIG. 6, the second computation unit 136 may also function as a medium amount calculation unit 172.

In order to reduce the burden on the user in the cell culturing operation, it may be preferable to reduce the number of steps of the actual work. For example, if the bag 22a is filled with a large amount of medium, the number of times the user replaces the bag 22a can be reduced. On the other hand, a quality assurance storage period may be set for the medium. Filling a large amount of medium in the bag 22a may cause increases in the amount of medium left in the bag 22a even after expiration of the storage period. The medium left after expiration of the storage period may be discarded. When the culture medium is prepared in an excessive amount as described above, the waste amount of the medium may increase and the cost may increase. Therefore, it may be preferable to adjust the amount of the medium to be prepared according to the operation schedule.

The medium amount calculation unit 172 may calculate the amount of the medium to be prepared. The medium amount calculation unit 172 may determine the flow rate of the first supply pump 102 on each day based on the operation process and may calculate the amount (first supply amount) of the medium supplied to the first circulation flow path 58 on each day. Similarly, the medium amount calculation unit 172 may determine the flow rate of the second supply pump 106 on each day based on the operation process and may calculate the amount (second supply amount) of the medium supplied to the second circulation flow path 62 on each day.

The medium amount calculation unit 172 may check the first supply amount of each day, the second supply amount of each day, and the day on which the worker does not work and may calculate the amount of culture medium to be filled in each bag 22a. The medium amount calculation unit 172 may calculate the amount of the medium to be prepared at the timing of replacement of the bag 22a so that the medium in the bag 22a does not run out on a day on which the operator does not work. The medium amount calculation unit 172 can also calculate the amount of the medium to be prepared at the time of replacement of the bag 22a, using the storage period, the capacity of the bag 22a, a margin of the amount of the medium prepared, and the like. The user can operate the input unit 130 to input each piece of information such as the storage period, the capacity of the bag 22a, the margin of the amount of the medium prepared, and the like to the scheduling unit 132.

In at least one example embodiment, in the series of processes illustrated in FIG. 4, the process of step S1 and the process after step S2 may be performed at different timing.

The process of step S1 illustrated in FIG. 4 may not be performed. In such instances, the process acquisition unit 146 may acquire a preset operation process from the second storage unit 138.

The user may operate the input unit 130 to input the date and time when the user does the actual work instead of inputting the date and time when the user does not do the actual work. In such instances, the condition acquisition unit 148 may determine that the date and time other than the date and time when the user does the actual work as the date and time when the user does not do the actual work.

In accordance with various aspects of the present disclosure, the present disclosure reduces the burden on the user by ensuring that the actual work does not occur at a date and time when the user cannot work or at a date and time when the user does not want to work.

In accordance with various aspects of the present disclosure, the user can grasp the timing and contents of the actual work in the cell culturing from the calendar 160.

If a large amount of medium is contained in the bag 22a attached to the cell culturing device 12, a large amount of medium in the bag 22a may be left in a room temperature environment for a long time reducing the quality of the medium. In accordance with various aspects of the present disclosure, the user can prepare the medium as necessary at a date and time other than the date and time when the user does not work reducing the amount of the medium of declining quality.

In accordance with various aspects of the present disclosure, since the amount of the medium to be prepared is calculated, the amount of the medium to be discarded can be reduced.

1. A scheduling apparatus for scheduling cell culturing in a cell culturing device, the scheduling apparatus comprising:
a process determination unit configured to determine a timing at which an actual work requiring human labor occurs; and
a schedule fixing unit configured to adjust a schedule of the cell culturing so that the timing at which the actual work requiring human labor occurs does not conflict with a working date and time of a user of the cell culturing device.

2. The scheduling apparatus of claim 1, wherein
the scheduling apparatus further includes a condition acquisition unit configured to acquire an operational condition that includes the working date and time of the user, and
the schedule fixing unit is configured to adjust the schedule of the operation process so that the operational condition acquired by the condition acquisition unit does not conflict with time at which the actual work requiring human labor occurs.

3. The scheduling apparatus of claim 2, wherein the operational condition includes one or more dates and times at which the user does not work or one or more dates and times at which a user does work.

4. The scheduling apparatus of claim 3, wherein
the schedule fixing unit determines the one or more dates and times at which the user does not work based on the operational condition, and
the scheduling fixing unit adjusts the schedule of the cell culturing to eliminate overlap between the one or more dates and times at which the user does not work and the timing at which the actual work requiring human labor occurs.

5. The scheduling apparatus of claim 2, wherein the scheduling apparatus further includes:
an input unit configured to receive input of the operational condition by the user, the condition acquisition unit acquiring the operational condition from the input unit.

6. The scheduling apparatus of claim 5, wherein
the scheduling apparatus further includes a process acquisition unit configured to acquire an operation process of the cell culturing, and
the schedule fixing unit is configured to adjust a schedule of the operation process so that the timing at which the actual work requiring human labor occurs does not conflict with the working date and time of the user.

7. The scheduling apparatus of claim 6, wherein the scheduling apparatus further includes:
a simulation execution unit configured to execute simulation of the cell culturing; and
a process creation unit configured to create the operation process based on a result of the simulation, the process acquisition unit acquiring the operation process from the process creation unit.

8. The scheduling apparatus of claim 6, wherein the actual work requiring human labor includes:
an attachment operation that includes attaching a container containing a liquid for the cell culturing.

9. The scheduling apparatus according to claim 8, wherein
the input unit is further configured to receive inputs of a capacity of the container,
the condition acquisition unit acquires the capacity of the container input by the input unit, and
the process determination unit is further configured to determine the timing at which the attachment operation of attaching the container occurs based on the capacity of the container acquired by the condition acquisition unit.

10. The scheduling apparatus according to claim 8, wherein the scheduling apparatus further includes:
a medium amount calculation unit configured to calculate an amount of medium to be prepared to fill the container at least based on the operational condition acquired by the condition acquisition unit and the operation process acquired by the process acquisition unit.

11. The scheduling apparatus according to claim 10, wherein
the input unit is further configured to receive inputs of a storage period of the medium, and
the medium amount calculation unit calculates the amount of the medium at least based on the operational condition, the operation process, and the storage period.

12. The scheduling apparatus of claim 1, wherein the scheduling apparatus further includes:
a display unit configured to display the schedule of the cell culturing as adjusted by the schedule fixing unit.

13. A scheduling method for scheduling cell culturing in a cell culturing device, the method comprising:
determining a timing at which an actual work requiring human labor occurs; and
adjusting a schedule of the cell culturing so that the timing at which the actual work requiring human labor occurs does not conflict with a working date and time of a user of the cell culturing device.

14. The method of claim 13, wherein
the method further includes acquiring operational condition that includes the working date and time of the user, and
the adjusting of the schedule of the cell culturing so that the operational condition acquired does not conflict with the time at which the actual work requiring human labor occurs.

15. The method of claim 14, wherein the acquiring of the operational conditions includes acquiring one or more dates and times at which the user does not work or one or more dates and times at which a user does work.

16. The method of claim 15, wherein the adjusting of the schedule of the cell culturing so that the timing at which the actual work requiring human labor occurs does not conflict with the working date and time of the use includes eliminating overlap between the one or more dates and times at which the user does not work and the timing at which the actual work requiring human labor occurs.

17. The method of claim 14, wherein
the method further includes acquiring an operation process of the cell culturing, and
the adjusting of the schedule of the cell culturing includes adjusting a schedule of the operation process so that the timing at which the actual work requiring human labor occurs does not conflict with the working date and time of the user.

18. The method of claim 17, wherein the method further includes:
simulating the cell culturing; and
creating the operation process based on a result of the stimulation.

19. The method of claim 13, wherein the actual work requiring human labor includes:
an attachment operation that includes attaching a container containing a liquid for the cell culturing.

20. The method of claim 13, wherein the cell culturing device includes a scheduling apparatus, the scheduling apparatus including:
a process determination unit configured to do the determining of the timing at which the actual work requiring human labor occurs;
a scheduling fixing unit configured to do the adjusting of the schedule of the cell culturing so that the timing at which the actual work requiring human labor occurs does not conflict with the working date and time of the user;
a condition acquisition unit configured to do the acquiring of the operational condition that includes the working date and time of the user; and
a process acquisition unit configured to do the acquiring of the operation process of the cell culturing.
